(19) United States
(12) Patent Application Publication
(10) Pub. No.: US 2011/0243913 A1
(43) Pub. Date: Oct. 6, 2011

(54) Biomaterial Compositions and Methods of Use

(75) Inventor: James San Antonio, Media, PA (US)

(73) Assignee: ORTHOVITA, INC.

(21) Appl. No.: 13/081,192

(22) Filed: Apr. 6, 2011

Related U.S. Application Data
(60) Provisional application No. 61/321,284, filed on Apr. 6, 2010.
(60) Provisional application No. 61/321,296, filed on Apr. 6, 2010.

(51) Int. Cl. A61K 33/42; A61K 38/45; A61P 19/08

(52) U.S. Cl. 424/94.5; 424/602

(57) ABSTRACT

The invention relates to biomaterial compositions and methods for promoting bone regeneration and hemostasis. The invention also relates to compositions and methods for promoting wound healing. In various embodiments, the compositions comprise crosslinkable collagen molecules and calcium phosphate suitable for bone regeneration. In various embodiments, the compositions comprise crosslinkable collagen molecules suitable for promoting hemostasis or wound healing; or suitable as tissue sealants. In some embodiments, the compositions contain additional agents, including biological agents.

### CROSS-REFERENCE TO RELATED APPLICATIONS

The present application claims the benefit of priority under 35 U.S.C. §119(e) to U.S. Provisional Application No. 61/321,284, filed on Apr. 6, 2010, and to U.S. Provisional Application No. 61/321,296, filed on Apr. 6, 2010, both of which applications are incorporated by reference herein in their entirety.

### BACKGROUND

There has been a continuing need for improved biomaterials, and particularly for improved resorbable bone regeneration and resorbable hemostat materials.

Bone Regeneration Materials

Although autograft materials have very good properties and radiopacity for bone regeneration procedures, their use exposes patients to the risk of second surgeries, pain, and morbidity at the donor site. Allograft devices, which are processed from donor bone, also have very good radiopacity, but carry the risk of disease transmission and the quality of the allograft devices varies because they are natural. Also, there tend to be limitations on supply.

In recent years, synthetic materials have become a viable alternative to autograft and allograft devices. One such synthetic material is Vitoss® Bone Graft Substitute (Orthovita, Inc., Malvern, Pa., assignee of the present application). Like autograft and allograft, synthetic graft materials serve as osteoconductive scaffolds that promote the ingrowth of bone. As bone growth is promoted and increases, the graft material resorbs and is eventually replaced with new bone.

Many synthetic bone grafts include materials that closely mimic mammalian bone, such as compositions containing calcium phosphates. Exemplary calcium phosphate compositions contain type-B carbonated hydroxyapatite, which is the principal mineral phase found in the mammalian body. The ultimate composition, crystal size, morphology, and structure of the body portions formed from the hydroxyapatite are determined by variations in the protein and organic content. Calcium phosphate ceramics have been fabricated and implanted in mammals in various forms including, but not limited to, shaped bodies and cements. Different stoichiometric compositions such as hydroxyapatite (HAp), tricalcium phosphate (TCP), tetracalcium phosphate (TTCP), and other calcium phosphate salts and minerals, have all been employed to match the adaptability, biocompatibility, structure, and strength of natural bone. The role of pore size and porosity in promoting revascularization, healing, and remodeling of bone has been recognized as a critical property for bone grafting materials. The preparation of exemplary porous calcium phosphate materials that closely resemble bone have been disclosed, for instance, in U.S. Pat. No. 6,383,519 (hereinafter the '519 patent“) and U.S. Pat. No. 6,521,246 (hereinafter the '246 patent”).

There has been a continuing need for improved bone graft systems. Although calcium phosphate bone graft materials are widely accepted, they lack the strength, handling and flexibility necessary to be used in a wide array of clinical applications. Heretofore, calcium phosphate bone graft substitutes have been used in predominantly non-load bearing applications as simple bone void fillers and the like. For more clinically challenging applications that require the graft material to take on load, bone reconstruction systems pair a bone graft material with traditional rigid fixation systems. The prior art discloses such bone reconstruction systems. For instance, MacroPore OS™ Reconstruction System is intended to reinforce and maintain the relative position of weak bony tissue such as bone graft substitutes or bone fragments from comminuted fractures. The system is a resorbable graft containment system composed of various sized porous sheets and sleeves, non-porous sheets and sleeves, and associated fixation screws and tacks made from polylactic acid (PLA). However, the sheets are limited in that they can only be shaped for the body when heated.

The Synthes SynMesh™ consists of flat, round, and oval shaped cylinders customized to fit the geometry of a patient's anatomical defect. The intended use is for reinforcement of weak bony tissue and is made of commercially pure titanium. Although this mesh may be load bearing, it is not made entirely of materials that are flexible and is not resorbable.

There remains a need for flowable, resorbable bone regenerative materials that have structural integrity and can take on load. Further, there remains a need for a flowable, resorbable bone regenerative material that can set after being injected, particularly when injected via minimally invasive procedures.

Hemostat Materials

As with currently available bone regeneration materials, currently used hemostatic materials also have limitations, particularly in surgical applications in which there is severe bleeding at the site. Further, many require the use of autologous fibrinogen and FXIII, together with exogenous collagen and thrombin. While the use of autologous fibrinogen avoids problems with rejection of the material, the production of these compositions can require relatively large amounts of the patient's blood and long preparation times. Moreover, the need to add exogenous thrombin makes these formulations very expensive.

There is also a need in the art for compositions that achieve hemostasis in a rapid manner and which avoid the requirement of exogenously added thrombin; and for hemostatic compositions that can be used to control severe bleeding.

The present invention biomaterial fulfills these needs.

### SUMMARY OF THE INVENTION

The present invention relates to compositions and methods for promoting bone regeneration and wound healing. In various embodiments, the compositions comprise crosslinkable collagen molecules or microfibrils suitable for bone regeneration, for promoting hemostasis or wound healing; or suitable as tissue sealants. In some preferred compositions for bone regeneration, the compositions also include calcium phosphate. In certain embodiments, the compositions contain additional agents, including biological agents.

The present invention is particularly suited for minimally invasive bone repair procedures that require a settable material. Heretofore, bone regeneration compositions that employ a “settable collagen” have not been known. Thus, the present invention material can be injected and remain at the implantation site to serve as a scaffold for bone regeneration; and then resorb over time after fulfilling the purpose of bone growth.

### BRIEF DESCRIPTION OF THE DRAWINGS

FIG. 1 depicts the results of an example experiment assessing the flow of calcium phosphate-collagen suspensions with and without transglutaminase (Tg) treatment. Calcium phosphate-collagen suspensions were prepared in glass test tubes and before incubation when placed horizontally, the material flowed along the test tube wall. Calcium phosphate-collagen suspensions with and without Tg addition were incubated vertically at 37° C. for 1 hour, and afterwards were placed horizontally on the bench top at ambient temperature and photographed at various time points.

FIG. 2 depicts the results of an example experiment assessing the extent of flow of untreated control and Tg-treated calcium phosphate-collagen suspensions in test tubes.

FIG. 3 depicts the results of an example experiment evaluating the extrusion of a collagen-thrombin mixture (“Vitagel” left) and calcium phosphate-collagen-thrombin suspension (“Vitoss-Vitagel” right). The mixtures were extruded from a 5.0 ml syringe into physiological saline in wells of a 35 mm culture dish, and photographed several hours later. Both materials were easily extruded and retained their rope-like forms.

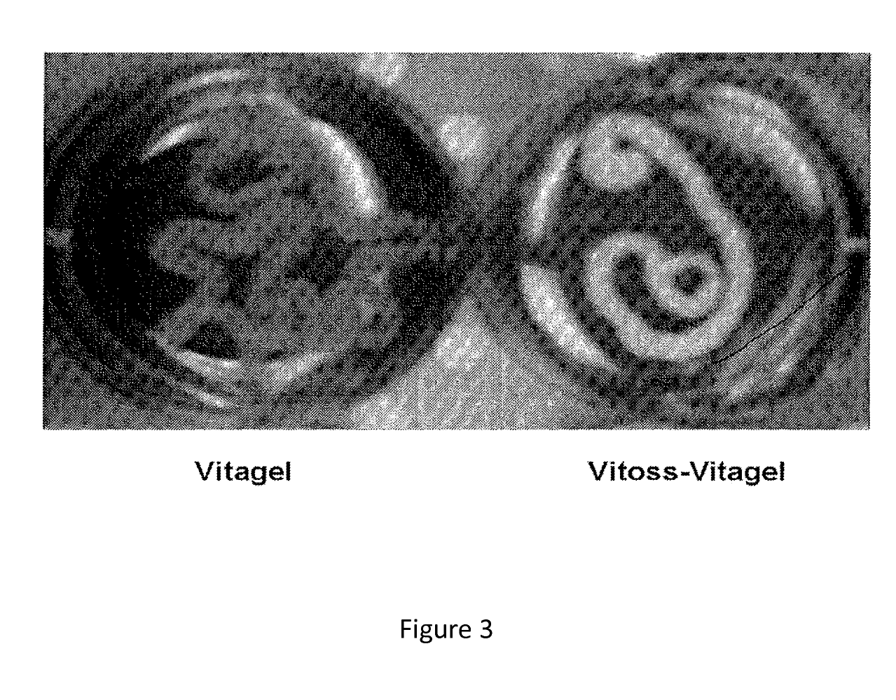

FIG. 4 depicts the results of an example experiment assessing the flow of calcium phosphate-collagen suspensions with and without transglutaminase (Tg) treatment. FIG. 4A. Bovine marrow bones were cleaned of marrow to about 2 cm deep to create bone marrow “cups” for sample incubation. FIG. 4B. Calcium phosphate—collagen suspensions without (control) (left) or with Tg treatment (right) were incubated vertically at 37° C. for 2 hours. FIG. 4C. After incubation bone segments were placed horizontally onto the lab bench at ambient temperature and within one minute the control material (left) had flowed completely out of the bone, but the Tg-treated sample (right) remained unchanged. FIG. 4D. At ten minutes the Tg-treated material was still retained in the bone segment. FIG. 4E. By thirty minutes about half of the Tg-treated sample had flowed partially along the inner bone surface but the remainder was retained by the marrow and bone surface, which persisted even at three and a half hours when the experiment was terminated.

FIG. 5 shows a schematic of one type of complex that may be employed in the present invention—a biotin-avidin complex. The biotin, a small water soluble B-complex vitamin, is mixed with the protein avidin to form a complex of such high affinity that the interaction is considered essentially irreversible.

FIG. 6 shows a schematic of one embodiment of the present invention in which microfibrillar collagen is covalently derivatized to contain several biotin molecules per collagen microfibril.

FIG. 7 shows a schematic in which the biotinylated microfibrillar collagen of FIG. 6 and avidin-containing solutions are used together to create the bone regeneration and hemostatic compositions of the present invention. The collagen component exhibits platelet binding and activation activities, and the avidin-collagen interaction creates a high affinity and rigid mesh to promote platelet and erythrocyte trapping and support clot formation and stabilization. The collagen-avidin interactions occur immediately, followed by the lower affinity interactions between collagen monomers as they polymerize into fibrils. Note that the calcium phosphate component of the bone regeneration composition is not shown, although it should be understood that the collagen/avidin composition may serve as a carrier and scaffold for the calcium phosphate material as described herein.

### DETAILED DESCRIPTION

The present invention relates to compositions and methods for promoting bone regeneration and wound healing. In various embodiments, the compositions comprise crosslinkable collagen molecules or microfibrils suitable for bone regeneration, for promoting hemostasis or wound healing; or suitable as tissue sealants. In some preferred compositions for bone regeneration, the compositions also include calcium phosphate. In certain of these embodiments, the calcium phosphate particles of the invention comprise particles as described in U.S. Pat. Nos. 6,383,519, 6,521,246, 7,189,263, 7,531,004 and 7,534,451. In some embodiments, the compositions contain additional agents, including biological agents.

The biomaterial compositions of the invention are conveniently formed by mixing at least two components prior to use. In embodiments particularly suitable for bone regeneration, at least one of the components comprises a suspension of calcium phosphate particles in a liquid of crosslinkable collagen and at least one of the components comprises a crosslinking agent. In various embodiments, the two components are mixed prior to application of the composition to the tissue or bone of a patient. Although it is not necessary, the components can be formulated to have concentrations that allow mixing of the components in substantially equal volumes to simplify the final preparation of the composition. In various embodiments, a multiple barrel (i.e., one, two, three, or more) syringe with a disposable mixing tip can be used. In other embodiments, the two components can be mixed together using two or more separate syringes, or the two components can be directly applied to the tissue or bone site using a spatula or other surgical tool.

The compositions of the invention can be used in a variety of applications where known surgical hemostats and sealants, bone cements and bone void fillers have been used.

The composition can be used, by way of non-limiting examples, as a surgical hemostat or sealant, a wound repair adhesive, a soft tissue augmentor and a soft tissue substitute. The sealant can also be used to a attach skin graft to a site without the use of sutures, or with a reduced number of sutures. The surgical hemostat may be applied in a number of ways determined by the particular trauma, surgical indication and/or therapeutic technique.

Collagen

Collagen, preferably hypoallergenic collagen, is present in the composition in an amount sufficient to thicken the composition and augment its cohesive properties. The collagen may be a telopeptide collagen or telopeptide collagen (e.g., native collagen). In addition to thickening the composition, the collagen acts as a macromolecular lattice or scaffold. This feature gives more strength and durability to the resulting composition/clot. In addition, the collagen must be able to enhance gelation and set in a surgical sealant/bone regenerative composition.

One form of collagen that is employed may be described as at least “near native” in its structural characteristics. In various embodiments, the collagen may be characterized as resulting in insoluble fibers at a pH above 5; unless crosslinked or as part of a complex composition (e.g., bone). In some embodiments, the collagen will generally consist of a minor amount by weight of fibers with diameters greater than 50 nm, usually from about 1 volume % to about 25 volume %, and there will be substantially little, if any, change in the helical structure of the fibrils. In preferred embodiments, the collagen is microfibrillar type I collagen. Other forms of collagen which are employed may include microfibrillar collagen mixed with denatured collagen, or gelatin, or a mixture of microfibrillar collagen and gelatin in varying proportions. Although collagen can take many forms: denatured and sometimes partially fragmented as in gelatin; monomeric with a native triple helical conformation as in procollagen; polymerized into a five-mer aggregate as in microfibrillar collagen; or polymerized into higher-ordered cable-like fibrils as in fibrillar collagen, in this invention a “collagen molecule” may be taken to describe any of these entities or molecular forms of collagen.

In preferred embodiments, the collagen is microfibrillar type I collagen. Microfibrillar collagen may have several advantages in the present invention applications. First, microfibrillar collagen has been shown to have strong platelet activating activity owing to its ability, via the presence of glycine-proline-hydroxyproline repeats and integrin binding sites in its triple helical domain, to ligate and activate platelet GPVI and α2β1 integrin receptors. Second, microfibrillar collagen assembles into collagen fibrils which provide a rigid, settable substrate and mesh-like network to support platelet adhesion and clot stabilization. Third, during clot dissolution and wound healing, microfibrillar and fibrillar collagen that it may form should persist and by virtue of its ability to bind cells and growth and differentiation factors, serves as an ideal substrate for tissue regeneration and bone growth.

In other embodiments the collagen may comprise microfibrillar or fibrillar collagen mixed with various concentrations of denatured collagen, or gelatin. In yet further embodiments of the present invention, the collagen may be comprised entirely of any concentration of gelatin.

In various embodiments, the collagen molecules described herein are covalently modified to possess sulfhydryl groups. In certain embodiments, the addition of sulfhydryl groups is helpful for promoting intermolecular and intramolecular associations. In its processed native form, type I collagen does not contain cysteine, an amino acid that has a sulfhydryl group side chain. Therefore, selected amino acid side chains on type I collagen can be covalently modified with cysteine, in various embodiments of the invention, using techniques known in the art for modifying collagen. (See 2011, He et al., Acta Biomater., 7:1084-1093; 2000, Myles et al., J Biomater Sci Polymer Ed, 44:69-86). In various embodiments, the cysteine modification of collagen confers upon the protein the ability to form disulfide bonds with itself and/or with other cysteine-containing proteins. In other embodiments, the cysteine modification of collagen is useful for tailoring the strength and versatility of collagen molecules described herein. Moreover, the cysteine modification of collagen, in certain embodiments, can confer mucoadhesive properties of the collagen molecules described herein. Mucosal surfaces are known to contain mucoproteins rich in cysteine, which spontaneously form disulfide bonds amongst themselves and other cysteine-containing proteins. Thus, in one non-limiting example, the cysteine-modified collagen molecules described here exhibit enhanced mucoadhesive properties, thereby increasing their usefulness as tissue sealants, for example, in numerous biomedical applications.

In various embodiments the collagen is in a physiologically acceptable liquid vehicle, such as an aqueous isotonic vehicle at about a physiologic salt concentration.

The amount of the collagen can be varied to provide formulations of differing viscosities and strengths, depending on the particular application. In some embodiments, the collagen is a flowable composition dispersed in phosphate buffered saline to provide a final concentration in the composition of at least about 5 mg/ml, preferably from about 5 mg/ml to about 50 mg/ml, more preferably from about 10 mg/ml to about 50 mg/ml, and most preferably from about 10 to about 40 mg/ml.

Crosslinkable Collagen and Crosslinking Agents

The collagen molecule preferably comprises at least one crosslinkable moiety that is able to form a bond, directly or indirectly, with another crosslinkable moiety on another collagen molecule. Any crosslinkable moieties known in the art may be used. By way of non-limiting examples, the collagen molecules can be crosslinked by covalent interactions, by non-covalent interactions, by thermally reversible interactions, by ionic interactions, or by combinations thereof. These moieties can be crosslinked by physical, chemical, thermal, or photointiation (e.g., visible, UV) means, or by any combination thereof.

In some embodiments, a transglutaminase is used to crosslink collagen molecules. Transglutaminases are known to catalyze the formation of covalent bonds between a free amine group on protein-bound lysines and the gamma-carboxamide group of protein-bound glutamines. Bonds formed by transglutaminase are highly resistant to proteolytic degradation. Non-limiting examples of transglutaminases useful in the compositions and methods of the invention include Factor XIII, a blood clotting cascade component, and Streptomyces mobaraensis transglutaminase (e.g., Activa TG™). In other embodiments, genipin or glutaraldehyde are used, alone or in combination with other crosslinkers, to crosslink collagen molecules in the compositions and methods of the invention.

In some embodiments the calcium phosphate and collagen components of a mixture are crosslinked after they are delivered into a bone defect; in other embodiments they are crosslinked during their delivery; still in other embodiments they are crosslinked before their delivery. In embodiments in which the composition is used as a hemostat, crosslinking of collagen may be carried out before use of the formulation for hemostasis; while in other embodiments, crosslinking may be carried out at the time of application to the patient.

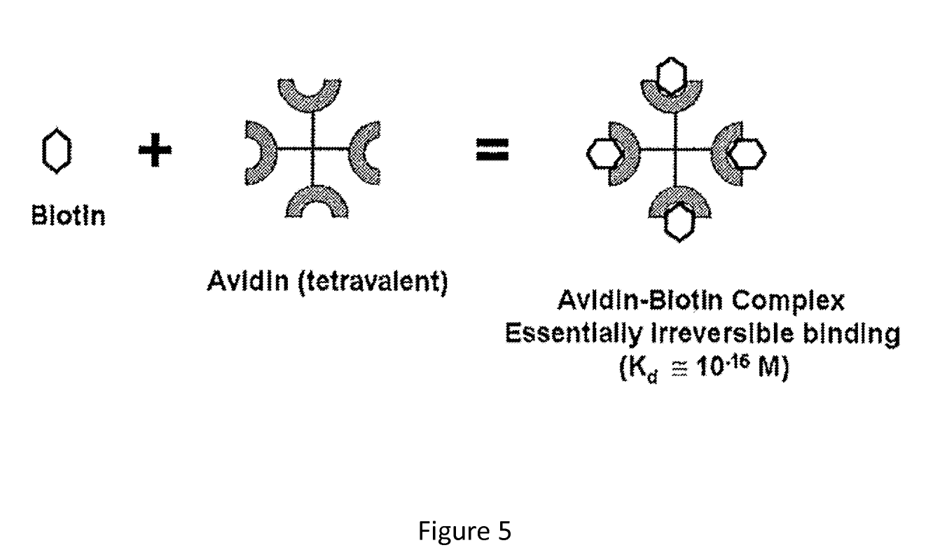

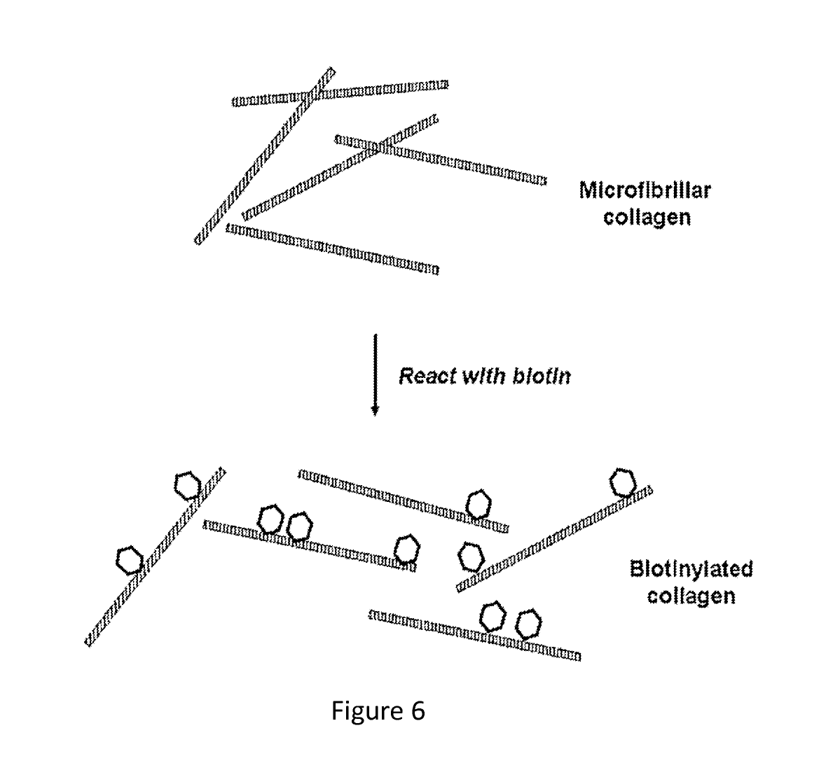

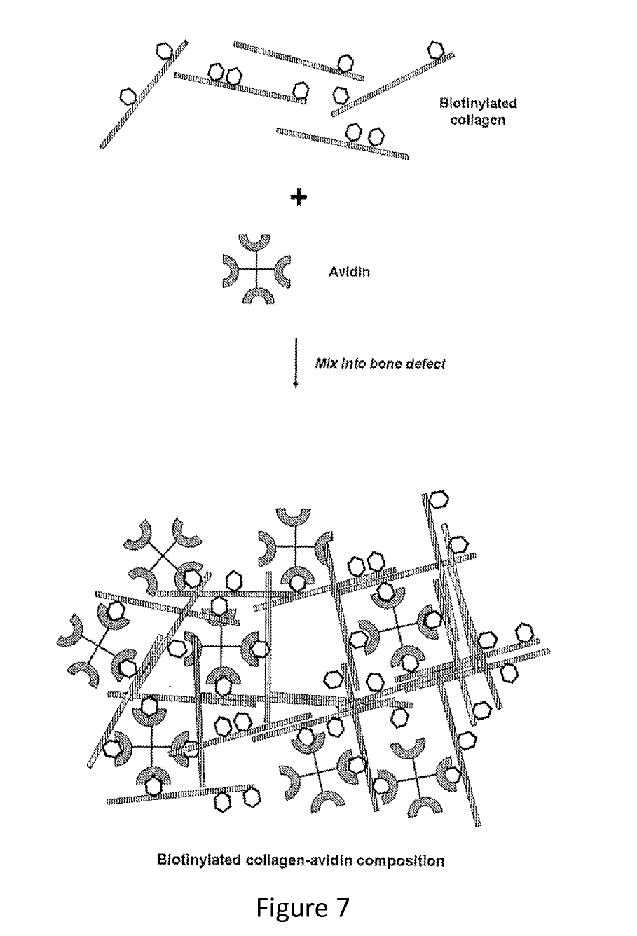

In preferred embodiments, an avidin-biotin interaction (FIG. 5) may be employed to connect collagen molecules to one another. In one embodiment, the collagen molecules are biotinylated and the biotinylated collagen molecules (FIG. 6) are then crosslinked using a biotin-binding protein, such as avidin (FIG. 7). Biotin can be attached to collagen by a variety of methods known in the art, including, by way of non-limiting example, the method reported by Lee at al. (2006, Mol Biol Cell 17: 4812-4826). In various embodiments, the number of biotin molecules attached to each collagen molecule can be adjusted to control the number of crosslinks. For example, type I collagen has side chains that can serve as sites for biotinylation, such as, but not limited to, the basic side chains of the amino acids lysine and arginine. Type I collagen is a triple helical monomer comprised of two alpha 1 chains and one alpha 2 chain, which contain 76 and 68 basic amino acids, respectively (1984, Miller, Chemistry of the collagens and their distribution, Chapter 2, p 41-81, in: Extracellular Matrix Biochemistry, Reddi A H, Piez K A, editors). Therefore, on average, the maximum number of biotin adducts that can occur on any collagen chain is about 73. The biotinylation reaction parameters (such as, but not limited to, the concentration of the collagen fibrils, the biotin concentration, the temperature, the reaction time, the ratio of collagen fibrils to biotin, etc.) can be modified to achieve the desired extent of collagen biotinylation of a collagen molecule.

In various embodiments, the number of biotin-binding molecules attached to each collagen molecule in the first population of collagen molecules ranges from about 1 to about 73, from about 1 to about 60, from about 1 to about 50, from about 1 to about 40, from about 1 to about 30, from about 1 to about 20, or from about 1 to about 10. In other embodiments, the number of biotin-binding molecules attached to each collagen molecule in the first population of collagen molecules ranges from about 2 to about 73, from about 2 to about 60, from about 2 to about 50, from about 2 to about 40, from about 2 to about 30, from about 2 to about 20, from about 2 to about 10, or from about 2 to about 4.

In various embodiments, the number of biotin molecules attached to each collagen molecule in the second population of collagen molecules ranges from about 1 to about 73, from about 1 to about 60, from about 1 to about 50, from about 1 to about 40, from about 1 to about 30, from about 1 to about 20, or from about 1 to about 10. In other embodiments, the number of biotin molecules attached to each collagen molecule in the second population of collagen molecules ranges from about 2 to about 73, from about 2 to about 60, from about 2 to about 50, from about 2 to about 40, from about 2 to about 30, from about 2 to about 20, from about 2 to about 10, or from about 2 to about 4.

Avidin is a 66-69 kDa tetrameric protein having four identical subunits, each of which can bind to biotin with high affinity and specificity. The dissociation constant of the avidin-biotin interaction is reported to be about K_{D}≈10⁻¹⁵ M, making it one of the strongest known non-covalent bonds. Biotin-binding molecules useful in the compositions and methods of the invention include, by way of non-limiting examples, avidin, streptavidin, tamavidin, NeutrAvidin™ and CaptAvidin™.

In another embodiment, a biotin-binding molecule is attached to each collagen molecule in a first population of collagen molecules and biotin is attached to each collagen molecule in a second population of collagen molecules so that when these two populations of collagen molecules are mixed together, each collagen molecule attaches to at least one other collagen molecule through one or more avidin-biotin interactions. In various embodiments, the number of biotin-binding molecules attached to each collagen molecule in the first population of collagen molecules ranges from about 1 to about 73, from about 1 to about 60, from about 1 to about 50, from about 1 to about 40, from about 1 to about 30, from about 1 to about 20, or from about 1 to about 10. In other embodiments, the number of biotin-binding molecules attached to each collagen molecule in the first population of collagen molecules ranges from about 2 to about 73, from about 2 to about 60, from about 2 to about 50, from about 2 to about 40, from about 2 to about 30, from about 2 to about 20, from about 2 to about 10, or from about 2 to about 4. In various embodiments, the number of biotin molecules attached to each collagen molecule in the second population of collagen molecules ranges from about 1 to about 73, from about 1 to about 60, from about 1 to about 50, from about 1 to about 40, from about 1 to about 30, from about 1 to about 20, or from about 1 to about 10. In other embodiments, the number of biotin molecules attached to each collagen molecule in the second population of collagen molecules ranges from about 2 to about 73, from about 2 to about 60, from about 2 to about 50, from about 2 to about 40, from about 2 to about 30, from about 2 to about 20, from about 2 to about 10, or from about 2 to about 4.

In yet another embodiment, a biotin-binding molecule is attached to each collagen molecule in a population of collagen molecules and biotin molecule is attached to each three-stranded β-sheet peptide molecule (see, for example, De Alba et al., 1999, Protein Sci 8:854-865) in a population of three-stranded β-sheet peptide molecules so that when these two populations of molecules are mixed together, each collagen molecule attaches to at least one three-stranded β-sheet peptide molecule through one or more avidin-biotin interactions and each three-stranded β-sheet peptide molecule attaches to at least two collagen molecules through two or more avidin-biotin interactions. In various embodiments, the number of biotin-binding molecules attached to each collagen molecule in the population of collagen molecules ranges from about 1 to about 73, from about 1 to about 60, from about 1 to about 50, from about 1 to about 40, from about 1 to about 30, from about 1 to about 20, or from about 1 to about 10. In other embodiments, the number of biotin-binding molecules attached to each collagen molecule in the population of collagen molecules ranges from about 2 to about 73, from about 2 to about 60, from about 2 to about 50, from about 2 to about 40, from about 2 to about 30, from about 2 to about 20, from about 2 to about 10, or from about 2 to about 4. In various embodiments, the number of biotin molecules attached to each three-stranded β-sheet peptide molecule in the population of three-stranded β-sheet peptide molecules ranges from about 1 to about 73, from about 1 to about 60, from about 1 to about 50, from about 1 to about 40, from about 1 to about 30, from about 1 to about 20, or from about 1 to about 10. In other embodiments, the number of biotin molecules attached to each three-stranded β-sheet peptide molecule in the population of three-stranded β-sheet peptide molecules ranges from about 2 to about 73, from about 2 to about 60, from about 2 to about 50, from about 2 to about 40, from about 2 to about 30, from about 2 to about 20, from about 2 to about 10, or from about 2 to about 4.

In still a further embodiment, a biotin molecule is attached to each collagen molecule in a population of collagen molecules and a biotin-binding molecule is attached to each three-stranded β-sheet peptide molecule in a population of three-stranded β-sheet peptide molecules so that when these two populations of molecules are mixed together, each collagen molecule attaches to at least one three-stranded β-sheet peptide molecule through one or more avidin-biotin interactions and each three-stranded β-sheet peptide molecule attaches to at least two collagen molecules through two or more avidin-biotin interactions. In various embodiments, the number of biotin molecules attached to each collagen molecule in the population of collagen molecules ranges from about 1 to about 73, from about 1 to about 60, from about 1 to about 50, from about 1 to about 40, from about 1 to about 30, from about 1 to about 20, or from about 1 to about 10. In other embodiments, the number of biotin molecules attached to each collagen molecule in the population of collagen molecules ranges from about 2 to about 73, from about 2 to about 60, from about 2 to about 50, from about 2 to about 40, from about 2 to about 30, from about 2 to about 20, from about 2 to about 10, or from about 2 to about 4. In various embodiments, the number of biotin-binding molecules attached to each three-stranded β-sheet peptide molecule in the population of three-stranded β-sheet peptide molecules ranges from about 1 to about 73, from about 1 to about 60, from about 1 to about 50, from about 1 to about 40, from about 1 to about 30, from about 1 to about 20, or from about 1 to about 10. In other embodiments, the number of biotin-binding molecules attached to each three-stranded β-sheet peptide molecule in the population of three-stranded β-sheet peptide molecules ranges from about 2 to about 73, from about 2 to about 60, from about 2 to about 50, from about 2 to about 40, from about 2 to about 30, from about 2 to about 20, from about 2 to about 10, or from about 2 to about 4.

Bridging Molecules

In various embodiments, the compositions of the invention further include a collagen bridging molecule. A collagen bridging molecule is any molecule that binds to a collagen monomer or fibril, and which is bi- or multivalent for collagen binding. As such, a collagen bridging molecule, when mixed with collagen, can bind to more than one collagen molecule and form a relatively stable, high or low affinity, non-covalent interaction. Collagen bridging molecules that exhibit a sufficiently high affinity interaction with collagen can promote collagen fibril network formation, stabilize clots, trap blood platelets, etc. In various embodiments, the collagen bridging molecule can be a protein or can be non-proteinaceous, such as glycosaminoglycans, or only in part proteinaceous, such as proteoglycans. In some embodiments, combinations of collagen bridging molecules can be used.

Collagen bridging molecules include any molecule that binds to collagen and is of sufficient length to span the distance between neighboring collagen molecules in a suspension, mixture, or formulation. Some collagen bridging molecules are multivalent for collagen binding in their native states, such as fibronectin, which is homodimeric. Other collagen bridging molecules are monovalent collagen binders in their native states, but can be converted to multivalent ligands if they are, by way of non-limiting examples, polymerized, covalently linked together, or altered in a recombinant protein form. Examples of collagen bridging molecules are described in DiLullo et al. (2002, J. Biol, Chem., 277:4223-4231) and Sweeney et al. (2008, J. Biol. Chem, 283:21187-21197), including glycosaminoglycans (e.g., heparin and chondroitin sulfates), fibronectin, cartilage oligomeric matrix protein (COMP), secreted protein acidic and rich in aspartic acid (SPARC), various integrin receptors (e.g., α1β1 and α2β1 heterodimers and their I-domains), matrix metalloproteinase-1 (MMP-1), proteoglycans (e.g., decorin), phosphophoryn, the platelet glycoprotein VI (GPVI) receptor, and Endo180.

In some embodiments, the collagen molecule is modified with a PEG chain. For example, the amine and carboxylic acid groups on collagen and gelatin are modified at a basic pH using PEG. Specifically, the length of the attached PEG chains can be adjusted to impart desired properties on the surface of the collagen molecules of the invention. In various embodiments, the number PEG chains attached to a collagen molecule, and the length of the PEG chains attached to the collagen molecule, can be adjusted to serve as a spacer to prevent non-specific or undesired protein adsorption and cell adhesion. In one embodiment, longer PEG chains can be used to limit protein adsorption and cell adhesion.

In various embodiments, the termini of the PEG chains are further modified with a bioactive agent. In various embodiments, the bioactive agent-modified termini of the PEG chains can signal through a particular receptor and trigger a cascade of reactions that will lead to a desired biological outcome such as, but not limited to, coagulation, osteoblast differentiation, etc.

In some embodiments, the end of a PEG chain is attached to collagen through a covalent linkage, while the non-reacted terminus of the PEG group possesses an attached biotin group. In some embodiments, the attached biotin group is used to crosslink collagen molecules using a biotin-binding molecule. In other embodiments, the attached biotin group is reacted with a bioactive agent, which is attached to a biotin-binding molecule. By altering the amount of biotinylated PEG that is attached to a collagen molecule, the extent of crosslinking, and the amount of bioactive agent presented on the collagen, can be tailored to achieve the desired level of crosslinking and bioactivity. In various embodiments, collagen molecules are crosslinked to the desired extent using a biotin-binding molecule, and then the remaining unreacted biotin groups on collagen are modified with bioactive agents bearing a biotin-binding moiety.

Biological Agents

A biological agent can be incorporated into the compositions of the invention. In some embodiments, the biological agent is mixed into a solution or suspension comprising the crosslinkable collagen. In such embodiments, the biological agent will be physically incorporated into the crosslinked collagen composition upon application. In other embodiments, the biological agent is incorporated by covalent or ionic attachment. In still further embodiments, the biological agent is incorporated in the form of a microsphere. Other agents may include agents having hemostatic activity.

Biological agents may be any of several classes of compound. Where the biological agents are proteins, peptides, or polypeptides, they may be derived from natural materials, or be materials produced by recombinant DNA technology, or mutant or engineered forms of natural proteins, peptides, or polypeptides, or produced by chemical modification of proteins, peptides, or polypeptides. The classes of biostatic agents listed herein, and the particular exemplars of each class, are to be considered as exemplary rather than limiting. Biological agents may, for example, be members of the natural coagulation pathway (“coagulation factors”). Such proteins include, by way of non-limiting examples, tissue factors, factors VII, VIII, IX, and XIII, fibrin, and fibrinogen.

A biological agent may also be a protein or other compound that activates or catalyzes the natural pathways of clotting (“coagulation activators”). These include, for example, thrombin, thromboplastin, calcium (e.g. calcium glucuronate), bismuth compounds (e.g. bismuth subgallate), collagen, desmopressin and analogs, denatured collagen (gelatin), and fibronectin. Vitamin K may contribute to activation of coagulation.

Further, a biological agent may be a particulate from the class of bioactive glasses such as 45S5 glass, Combeite glass-ceramic (Na₂O—CaO—P₂O₅—SiO₂), borate bioactive glass or combinations thereof. These fillers may possess a variety of morphologies such as, but not limited to, needles, particulate, flakes, cylinders, long fibers, whiskers, or spherical particles. In preferred embodiments, the filler is comprised of particles with an average particle size ranging from less than about 1.0 μm up to a range of from 2 to 3 millimeters (mm). Preferably, the average particle size distribution ranges from 1 to 100 μm. The particles may be of a single size within the above noted range or may be bimodal (of two different particle sizes within the range), trimodal, etc.

A biological agent may act by activating, aggregating or stimulating platelets (“platelet activators”), including, for example, cycloheximide, N-monomethyl L-arginine, atrial naturetic factor (ANF), small nucleotides (including cAMP, cGMP, and ADP), prostaglandins, thromboxanes and analogs thereof, platelet activating factor, phorbols and phorbol esters, ethamsylate, and hemoglobin. Nonabsorbable powders such as talc, and denatured or surface-absorbed proteins can also activate platelets.

A biological agent may act by local vasoconstriction (“vasoconstrictors”), such as, by way of non-limiting examples, epinephrine (adrenaline), adrenochrome, tetrahydrozoline, antihistamines (including antazoline), oxymetazoline, vasopressin and analogs thereof, and cocaine.

A biological agent may act by preventing destruction or inactivation of clotting reactions (“fibrinolysis inhibitors”), including, by way of non-limiting examples, eosinophil major basic protein, aminocaproic acid, tranexamic acid, aprotinin (Trasylol™), plasminogen activator inhibitor, plasmin inhibitor, alpha-2-macroglobulin, and adrenoreceptor blockers.

Thrombin acts as a catalyst for fibrinogen to provide fibrin, an insoluble polymer. In some embodiments, thrombin is present in the composition in an amount sufficient to catalyze polymerization of fibrinogen present in a patient's plasma. Thrombin also activates FXIII, a plasma protein that catalyzes covalent crosslinks in fibrin, rendering the resultant clot insoluble. In less preferred embodiments, the thrombin is present in the tissue adhesive composition in a concentration of from about 0.01 to about 1000 or greater NIH units (NIHu) of activity, preferably about 1 to about 500 NIHu, and more preferably about 200 to about 500 NIHu.

The fibrinogen, thrombin, FXIII or other natural protein used in the composition may be substituted by other naturally occurring or synthetic compounds or compositions which fulfill the same functions, e.g. a reptilase coagulation catalyzed, for example, ancrod, in place of thrombin.

In some embodiments, the composition of the invention will additionally comprise an effective amount of an antifibrinolytic agent to enhance the integrity of the clot as the healing process occurs. A number of antifibrinolytic agents are well known and include aprotinin, C1-esterase inhibitor and ε-amino-n-caproic acid (EACA). EACA is effective at a concentration of from about 5 mg/ml to about 40 mg/ml of the final adhesive composition, more usually from about 20 to about 30 mg/ml. EACA is commercially available as a solution having a concentration of about 250 mg/ml. Conveniently, the commercial solution is diluted with distilled water to provide a solution of the desired concentration.

Other biological factors of interest include EGF, TGF-α, TGF-β, TGF-I and TGF-II, FGF, PDGF, IFN-α, IFN-β, IL-2, IL-3, IL-6, hematopoietic factor, immunoglobulins, insulin, corticosteroids, hormones,

In some embodiments, the composition contains at least one antibiotic. The therapeutic dose levels of a wide variety of antibiotics for use in drug release systems are well known. See for example, Collagen, 1988, Vol. III, Biotechnology; Nimni, (Ed.), CRC Press, Inc., pp. 209-221 and Biomaterials, 1980, Winter et al., (Eds.), John Wiley & Sons, New York, pp. 669-676. Anti-microbial agents are particularly useful for compositions applied to exposed wound repair sites such as sites in the mouth or to compromised wound sites such as burns.

A biological agent may comprise non-protein polymers that act to viscosify or gel, by interaction with proteins, by tamponnade, or by other mechanisms. Examples include oxidized cellulose, “Vicryl” and other polyhydroxyacids, chitosan, alginate, polyacrylic acids, pentosan polysulfate, carrageenan, and polyorthoesters (e.g., Alzamer).

A biological agent may be a material that forms a barrier to leakage by mechanical means not directly related to the natural clotting mechanisms (“barrier formers”). These include oxidized cellulose, ionically or hydrogen-bond crosslinked natural and synthetic polymers including chitin, chitosan, alginate, pectin, carboxymethylcellulose, and poloxamers, such as Pluronic surfactants.

Kits

The invention also includes a kit comprising a hemostatic tissue/bone regenerative composition as elsewhere described herein, and an instructional material which describes, for example, applying the hemostatic/bone regenerative composition of the invention, to the tissue/bone of a subject. Optionally, the kit comprises an applicator for administering the hemostatic tissue/bone regenerative composition. The kit may include the components in containers or multiple barrel syringes with a disposable mixing tip. For example, in one embodiment, the kit may include two pre-filled syringes in which one syringe contains a suspension of calcium phosphate- and collagen-containing particles suspended in biotinylated collagen and a second syringe contains an avidin suspension. Alternatively, the kit could include two pre-filled syringes in which one syringe contains a suspension of calcium phosphate- and biotinylated-collagen-containing particles in a biotinylated collagen solution, and a second syringe contains a suspension of avidin. In other embodiments the kit may contain three pre-filled syringes in which one syringe contains a suspension of calcium phosphate and biotinylated-collagen-containing particles, a second syringe contains either biotinylated or non-biotinylated collagen, and the third syringe contains a suspension of avidin. Other variants of such kits containing calcium phosphate and/or collagen, biotinylated collagen, and avidin suspensions and solutions can be envisioned.

Definitions:

The definitions used in this application are for illustrative purposes and do not limit the scope of the invention.

The articles “a” and “an” are used herein to refer to one or to more than one (i.e. to at least one) of the grammatical object of the article. By way of example, “an element” means one element or more than one element.

“Crosslink” is used to refer to the joining of at least two molecules (such as, for example, collagen), to each other by at least one physical or chemical means, or combinations thereof.

The terms “diminish” and “diminution,” as used herein, means to reduce, suppress, inhibit or block an activity or function by at least about 10% relative to a comparator value. Preferably, the activity is suppressed, inhibited or blocked by 50% compared to a comparator value, more preferably by 75%, and even more preferably by 95%.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

An “individual,” as that term is used herein, includes a member of any animal species. Such animal species include, but are not limited to, birds, humans and other primates, and other mammals including commercially relevant mammals such as cattle, pigs, horses, sheep, cats, and dogs.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Experimental Example 1

### Collagen Biotinylation Feasibility Studies

In order to evaluate the feasibility of the present invention, collagen was biotinylated using a biotinylation kit in accordance with the manufacturer's instructions.

It was demonstrated that collagen fibrils could be modified with biotin adducts. Thus, microfibrillar collagen was phosphate precipitated to yield insoluble native-type collagen fibrils. The fibril suspension was diluted to three concentrations and each was subjected to biotinylation using the Thermo Scientific EZ-link NHS-PEG4 kit. The collagen fibril samples were assayed for biotin content using a spectrophotometric assay provided in the biotinylation kit. It was demonstrated that collagen fibrils were biotinylated and that the extent of biotin adduct formation was inversely related to collagen concentration (Table 1).

TABLE 1
Biotinylation of type 1 collagen fibril suspensions

| | Biotin adducts per |
|---|---|
| Collagen fibrils (mg/ml) | collagen alpha chain (average)^{#,} * |
| 20 | 1.4 |
| 2.0 | 5.3 |
| 0.2 | 37.5 |

#Biotinylations were carried out with 13 μl of biotin and 2 hour reaction times.
*Values are the average of data from two experiments.

Note that since the native collagen molecule is triple helical (i.e., has three alpha chains), that the collagen monomers in these experiments actually contain approximately 4, 16, and 113 biotin adducts.

The data shown here demonstrate that the number of biotin adducts per collagen chain can be varied by altering the concentration of collagen fibrils in the reaction. The skilled artisan will understand that the number of biotin adducts per collagen chain can also be varied by altering other parameters of the reaction (e.g., the biotin concentration, the temperature, the reaction time, the ratio of collagen fibrils to biotin, etc.).

### Experimental Example 2

### Calcium Phosphate-Collagen Mixtures: Flow and Effect of Transglutaminase

Experiments were conducted to determine whether small particles of calcium phosphate suspended in microfibrillar collagen solutions exhibit flow and whether crosslinking of the collagen molecules with transglutaminase would affect their flow.

The materials and methods used in this example are now described.

Materials

A mixture of microfibrillar collagen and thrombin was used as a source of microfibrillar collagen. Vitoss® calcium phosphate particles of approximately <1.0 mm (1000 μm) in diameter were used, Microbial transglutaminase (Activa™ RM; Ajinomoto) was purchased from a commercial vendor. Bovine marrow bone segments (˜5 cm long) were obtained from Acme Market, Paoli, Pa.

Vitoss® Calcium Phosphate Preparation

Beta-tricalcium phosphate (β-TCP) particles were sieved into fractions enriched in <53, 53-200, 200-500, and 500-1000 μm diameter particles using a series of fine wire screens of defined mesh sizes with a Gilford sieving machine.

Bone Preparation

Marrow bone segments were cleaned using a metal spatula to remove approximately half of the marrow to create bone “cups” into which calcium phosphate-collagen suspensions could be poured and incubated.

Preparation of Calcium Phosphate-Collagen Mixtures

A commercial grade mixture of collagen and thrombin (collagen/thrombin suspension portion of Vitagel® Surgical Hemostat) was extruded from graduated plastic syringes into plastic reagent weigh boats in approximately 2.0 or 4.0 ml aliquots. The calcium phosphate particles (either 250-500 or 500-1000 μm) were weighed on an analytical balance and manually mixed at an approximately 1:8 (w/v) ratio into the mixture of collagen and thrombin in the plastic weigh boats using a disposable plastic pipette until they appeared to be homogeneous suspensions. The calcium phosphate-collagen suspensions were then poured into glass test tubes or marrow bone segments at 2.0 or 4.0 ml volumes, respectively.

Transglutaminase Treatment

The enzyme powder was brought to room temperature, weighed on an analytical balance, and mixed into the calcium phosphate-collagen suspensions at enzyme concentrations of approximately 1.0-5.0% using a disposable plastic pipette.

Sample Incubation

Test tubes or marrow segments containing the calcium phosphate-collagen suspensions were incubated vertically at 37° C. for 1-2 hours in plastic racks (for test tubes) or a covered plastic tray (for bone segments) in a temperature controlled, lidded water bath.

Flow Assays

After incubation, the calcium phosphate-collagen suspensions were removed from the water bath and examined for gravity-induced flow after placing them horizontally onto the lab bench surface at ambient temperature. Flow was assessed qualitatively, by photographing the mixture at various time intervals. Flow was also assessed quantitatively in some test tube samples by marking the position of the sample meniscus in tubes held vertically, and then using a ruler to measure the distance the sample moved from the meniscus over time after the tube had been placed horizontally.

The results of this experiment are now described.

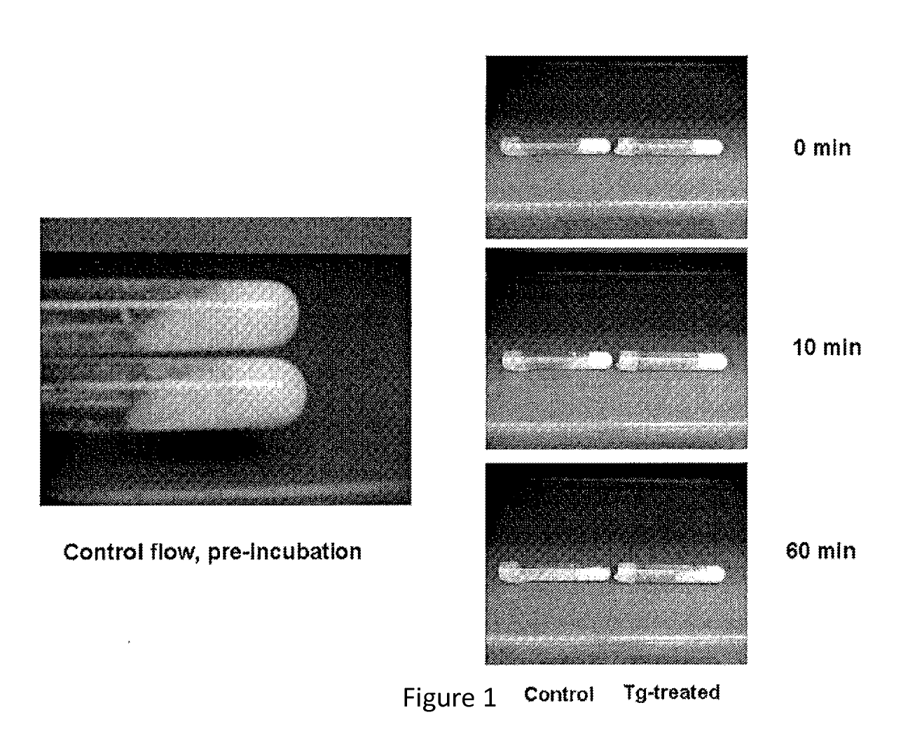

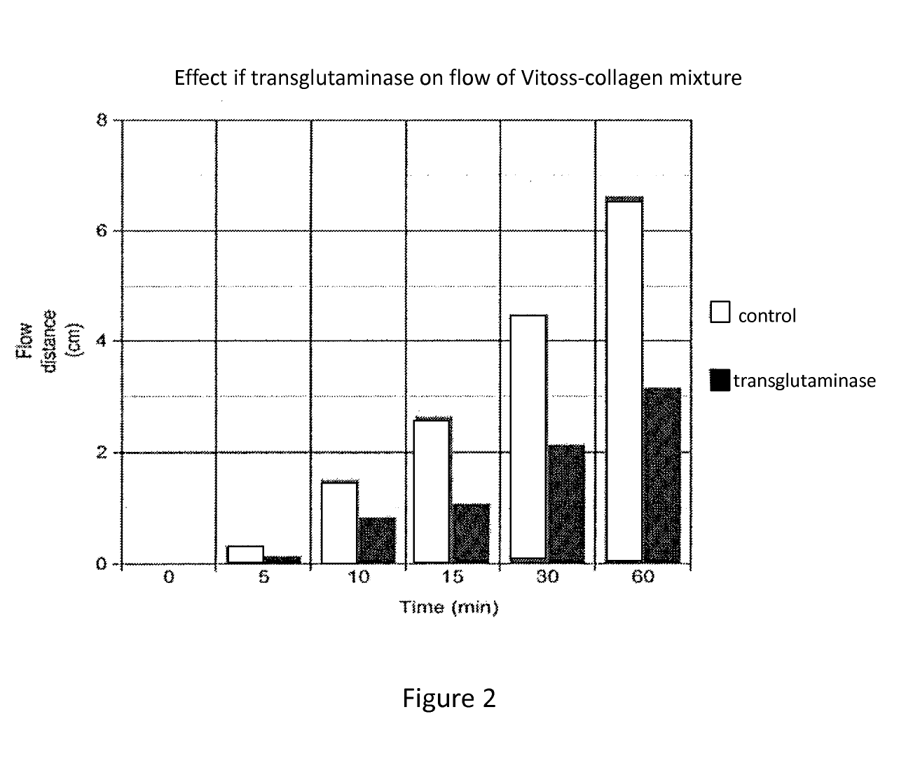

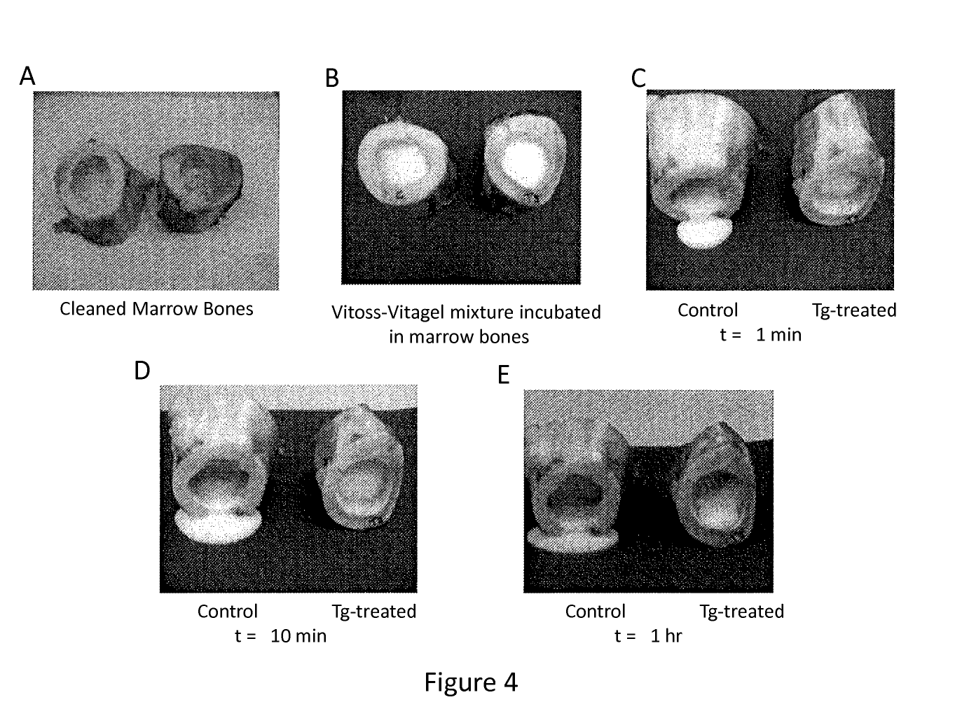

A 1:8 ratio suspension of the calcium phosphate particles to the collagen solution was easily prepared and exhibited gravity-induced flow. Thus, samples could be poured from vessels, or manually pushed using a spatula, from a plastic weigh boat into a tube (FIG. 1) or into a bone segment (FIG. 4). These mixtures appeared homogenous even after several hours, i.e., the calcium phosphate particles did not appear to settle out of the suspension, even when no transglutaminase treatment was carried out (FIGS. 1 and 4). When the mixture was placed in a 5.0 ml plastic syringe, it could easily be extruded into a saline solution, where it formed a rope-like stream which held its shape on a macro scale for many hours (FIG. 2).

When the calcium phosphate-collagen suspensions were incubated in glass test tubes under control conditions and then placed horizontally, they initially resisted flow (FIGS. 1 and 2), but within a few minutes the material deformed and the solution began to flow towards the top of the tube (FIGS. 1 and 2). By about one hour, the control solution had flowed along the full length of the tube. In contrast, transglutaminase-treated mixtures resisted flow for a longer period of time and by one hour had traveled only about half the distance of the controls (FIGS. 1 and 2).

In one experiment bone segments were partially cleaned of their marrow (FIG. 4A), then filled with either control or transglutaminase-treated calcium phosphate-collagen suspensions and were incubated vertically (FIG. 4B). After incubation, the bones were place horizontally. The control solution immediately flowed out of the bone (FIG. 4C). In contrast, the transglutaminase-treated solution was retained in its original position until about ten to fifteen minutes, and then it began to deform and flow (FIG. 4D). By thirty to sixty minutes, about half of the transglutaminase-treated mixture had flowed along the bone's inner surface, yet the rest appeared to remain adherent to the marrow and inner bone surface (FIG. 4E), which persisted until the experiment was terminated at three and a half hours. Throughout that time none of the transglutaminase-treated calcium phosphate-collagen suspension flowed out of the bone, as the control mixture had done within the first minute of the experiment.

In summary, a 1:8 calcium phosphate particle:collagen mixture was observed to flow and was capable of being extruded from a syringe. Moreover, transglutaminase treatment significantly reduced the flow of the calcium phosphate-collagen suspension from glass or marrow bone vessels. These results were seen for all control (n=5) and transglutaminase-treated (n=5) samples.

### Experimental Example 3

### Extrusion of Calcium Phosphate-Collagen Mixtures

Varying sizes and amounts of Vitoss® calcium phosphate particles of approximately <1.0 mm (1000 μm) were mixed with a microfibrillar collagen solution according to the table below. Each formulation was thoroughly mixed with a plastic transfer pipette in a plastic weigh boat and added to a standard BD 5 cc syringe. The ability of the formulation to be extruded through the Luer-lock tip of the syringe was assessed by visual inspection, as was its ability to maintain its shape for approximately 30 min after extrusion.

TABLE 2

| | Calcium | | |
|---|---|---|---|
| | Phosphate | Mass to Volume | |
| Collagen | (CaP) | Ratio (CaP: | |
| Solution | Particle Size | Collagen Solution) | Results |
| 20 mg/ml | <1 mm | 3:1 | Failed to extrude |
| 20 mg/ml | 200-500 μm | 3:1 | Failed to extrude |
| 20 mg/ml | 53-200 μm | 3:1 | Extruded easily; failed to |
| | | | maintain shape after |
| | | | extrusion |
| 20 mg/m1 | 53-200 μm | 2:1 | Extruded easily; maintained |
| | | | shape moderately well |
| 20 mg/ml | 53-200 μm | 1.5:1 | Extruded easily; maintained |
| | | | shape very well |
| 20 mg/m1 | 53-200 μm | 1:1 | Failed to mix well, formed a |
| | | | crumbly, non-homogenous |
| | | | mixture; failed to extrude |

### Other Embodiments

While the invention has been disclosed with reference to specific embodiments, it is apparent that other embodiments and variations of the invention may be devised by others skilled in the art without departing from the true spirit and scope of the invention. The appended claims are intended to be construed to include all such embodiments and equivalent variations.

The disclosures of each and every patent, patent application, and publication cited herein are hereby incorporated herein by reference in their entirety.

1. A bone regenerative composition comprising a suspension of calcium phosphate and collagen and a crosslinking agent.

2. The bone regenerative composition of claim 1 wherein the crosslinking agent comprises transglutaminase.

3. A bone regenerative composition comprising two components, wherein the first component is a suspension of calcium phosphate suspended in a population of collagen molecules and the second component is a population of biotin-binding molecules, and wherein at least one biotin molecule is attached to each collagen molecule in the population of collagen molecules.

4. The composition of claim 3, wherein when the two components are mixed together each collagen molecule in the population of collagen molecules attaches to at least one biotin-binding molecule and each biotin-binding molecule attaches to at least two collagen molecules through at least two biotin-binding molecule—biotin molecule interactions.

5. The composition of claim 3, wherein the biotin-binding molecule is at least one selected from the group consisting of avidin, streptavidin, and tamavidin.

6. The composition of claim 3, wherein the composition additionally comprises at least one biological factor.

7. The composition of claim 3, wherein the composition additionally comprises at least one crosslinking agent.

8. The composition of claim 7, wherein the crosslinking agent is at least one selected from the group consisting of genipin, glutaraldehyde and transglutaminase.

9. The composition of claim 3, wherein the composition additionally comprises at least one collagen bridging molecule.

10. A bone regenerative composition comprising two components, wherein the first component is a suspension of calcium phosphate suspended in a population of collagen molecules and the second component is a population of three-stranded β-sheet peptide molecules, and wherein at least one biotin molecule is attached to each collagen molecule in the population of collagen molecules, and wherein at least one biotin-binding molecule is attached to each three-stranded β-sheet peptide in the population of three-stranded β-sheet peptide molecules.

11. The composition of claim 10, wherein when the two components are mixed together each collagen molecule in the population of collagen molecules attaches to at least one three-stranded β-sheet peptide and each three-stranded β-sheet peptide attaches to at least two collagen molecules through at least two biotin-binding molecule—biotin molecule interactions.

12. The composition of claim 10, wherein the biotin-binding molecule is at least one selected from the group consisting of avidin, streptavidin, and tamavidin.

13. The composition of claim 10, wherein the composition additionally comprises at least one biological factor.

14. The composition of claim 10, wherein the composition additionally comprises at least one crosslinking agent.

15. The composition of claim 14, wherein the crosslinking agent is at least one selected from the group consisting of genipin, glutaraldehyde and transglutaminase.

16. The composition of claim 10, wherein the composition additionally comprises at least one collagen bridging molecule.

17. A method of forming a bone regenerative composition on a bone surface, the method comprising the steps of: applying the first component and the second component of the bone regenerative composition of claim 3 to the bone surface and allowing the collagen molecules therein to attach through biotin-binding molecule—biotin molecule interactions.

18. A method of forming a bone regenerative composition on a bone surface, the method comprising the steps of: applying the first component and the second component of the bone regenerative composition of claim 10 to the bone surface and allowing the collagen molecules therein to attach through biotin-binding molecule—biotin molecule interactions.
